(19) United States
(12) Patent Application Publication
(10) Pub. No.: US 2012/0121715 A1
(43) Pub. Date: May 17, 2012

(54) DNA Vaccine Comprising IL-6-Encoding DNA Construct and Applications Thereof

(75) Inventors: Wen-Fang Chang, Taipei (TW); Chi-An Chen, Taipei (TW)

(73) Assignee: NATIONAL TAIWAN UNIVERSITY, Taipei City (TW)

(21) Appl. No.: 13/351,726

(22) Filed: Jan. 17, 2012

Related U.S. Application Data
(63) Continuation-in-part of application No. 12/003,805, filed on Jan. 2, 2008, now Pat. No. 8,124,591.

(51) Int. Cl. A61K 9/14; A61P 11/00; A61P 35/00; A61P 31/12; C12N 15/63; A61K 39/12

(52) U.S. Cl. 424/490; 435/320.1; 424/204.1

(57) ABSTRACT

A DNA vaccine, which comprises a DNA construct comprising an expression vector which is expressible in a eukaryotic cell, and a nucleotide fragment which comprises an IL-6-encoding sequence and an HPV E7(detox)-encoding sequence. A pharmaceutical composition and a method for preventing or treating HPV-related (-induced) diseases are included.

This application is a continuation-in-part of U.S. application Ser. No. 12/003,805, filed Jan. 2, 2008, currently pending.

### BACKGROUND OF THE INVENTION

1. Field of the Invention

The present invention relates to a DNA vaccine comprising a DNA construct. The present invention also relates to a pharmaceutical composition and a method for preventing or treating HPV-related diseases.

2. Description of the Related Art

Cervical cancer is a main cause of death in women, and persistent infection with known high-risk Human papillomavirus (HPV) types, such as HPV subtype 16 (HPV-16) is directly linked to development and progression of cervical cancer. Although the present HPV vaccines can be used to prevent cervical cancer, it is not yet known whether they are useful for treating HPV-associated cervical cancer or ameliorating the existing HPV infection or HPV-associated lesions. Therefore, it is advantageous to develop a safe and effective therapeutic vaccine for treatment of HPV-associated cervical cancer, HPV-associated other genital cancers (e.g., vaginal, vulvar, penile), or other HPV-related cancers (e.g., lung cancer, esophageal cancer, head and neck cancer, throat cancer), and other HPV-associated lesions of head and neck, and gastrointestinal system, as well as for the treatment of precancerous lesions of the same areas.

In recent years, DNA vaccines have been developed and evaluated in a variety of disease models as promising therapeutic agents for treating a variety of diseases including their use as immunotherapy in certain cancers. Therapeutic DNA vaccines are introduced into antigen-presenting cells (APCs) of the recipient's immune system to express a protein antigen, the protein antigen is processed and presented by the major histocompatibility antigen (MHC) class I and II molecules to induce immune responses, such as helper T-cell response, cytotoxic T-cell response and humoral (antibody) response, and a direct result is that tumor cells expressing said antigen can be eliminated by the immune system.

In comparison with “traditional” (protein antigen) vaccines, DNA vaccines have many advantages such as high specificity, safety, stability, cost-effectiveness, and the ability to induce several types of immune responses. DNA vaccines do not pose any risk of infection because only certain sequences of the pathogen's DNA is used in their manufacture, in contrast to “live” and “attenuated” vaccines. Also, no toxic adjuvants are needed for practical use of DNA vaccines. DNA vaccines are also easier to prepare than “subunit” vaccines because no protein antigens need to be expressed and purified prior to injection into the patient.

However, because the lifespan of APCs is limited, the efficacy of DNA vaccines is limited because APCs are not able to process and present the antigen indefinitely. Therefore, several strategies have been applied to increase the potency of DNA vaccines, such as targeting an antigen by fusing molecules to enhance antigen processing (Cheng et al., 2001; Chen et al., 2000), targeting antigens for rapid intracellular degradation (Rodriguez et al., 1997), directing antigens to APCs by fusion to ligands for APC receptors (Boyle et al., 1998) or to a pathogen sequence such as fragment C of tetanus toxin (King et al., 1998), co-injecting cytokines (Weiss et al., 1998), and administering with CpG oligonucleotides (Klinman et al., 1997).

Combined strategies for enhancing the effects of DNA vaccines have been introduced in the development of cancer vaccines and immunotherapy, and overexpression of an anti-apoptotic molecule is a potential strategy for overcoming the short lifespan of APCs. For example, the administration of a DNA vaccine which comprises fragments encoding an antigen and an inhibitor of apoptosis of dendritic cells (DCs) can prolong the survival of DCs, thereby enhancing the potency of the DNA vaccine (Kim et al., 2003). Other research has demonstrated that combining a tumor antigen and an apoptosis inhibitor, such as Bcl-Xl, Bcl-2, XIAP, dominant negative caspase-9, or dominant negative caspase-8, can enhance the antigen-specific immunity and the anti-tumor effects (Kim et al., 2003; Kim et al., 2004; Kim et al., 2005). Thus, DC-based vaccination could be enhanced by an approach that inhibits apoptosis and prolongs the survival of antigen-expressing DCs in vivo. However, some apoptosis inhibitors, such as proteins of Bcl-2 family, are known to be over-expressed in some cancers and therefore they are implicated in contributing to cellular immortalization. This safety issue cannot be ignored.

### SUMMARY OF THE INVENTION

In view of the disadvantages of conventional DNA vaccines, the objective of the present invention is to develop a DNA vaccine having an improved potency, which comprises a sequence obtained from the human interleukin-6 (IL-6) gene and a sequence obtained from E7 gene (detox) of Human Papillomaviruses. The E7 gene with mutants in the critical retinoblastoma (Rb) binding residues (termed E7(detox)) is generated which failed to bind Rb, had no transforming activity, and yet maintained potent immunogenicity. The combination of IL-6 and E7(detox) prolongs the life of DCs, improves the processing and presentation of the E7 antigen, and enhances the recipient's immune responses.

Another objective of the present invention is to provide a pharmaceutical composition comprising said DNA vaccine and an optional pharmaceutically acceptable carrier. Yet another objective of the present invention is to provide a method for preventing or treating a HPV-related disease.

To achieve the above-mentioned objectives, the present invention provides a DNA construct, consisting of an expression vector, which is expressible in a eukaryotic cell; an IL-6-encoding sequence; and a human papillomavirus E7(detox) (E7(detox))-encoding sequence). The IL-6-encoding sequence consists of SEQ ID NO: 3. The encoded amino acid of the HPV E7(detox)-encoding sequence presents an original HPV-E7 encoded amino acid with mutations or deletions at positions 24, 25 or 26 which disrupt the Rb binding site.

In preferred embodiments, the encoded amino acid of the HPV E7(detox)-encoding sequence presents the HPV-E7 encoded amino acid with mutations at positions 24 and 26. The HPV E7(detox)-encoding sequence consists of SEQ ID NO: 6.

In preferred embodiments, said expression vector is expressible in human cells; more preferably, said expression vector is selected from pcDNA3, pSG5, or pCMV; and most preferably, said expression vector is pcDNA3.

The present invention also provides a DNA vaccine, comprising: the above-mentioned DNA construct.

In preferred embodiments, the DNA vaccine may further comprise a particle coated with said DNA construct. Said particle is a gold particle; more preferably, said gold particle has a diameter in the range of 1.6 μm.

In preferred embodiments, said expression vector is expressible in human cells; more preferably, said expression vector is selected from pcDNA3, pSG5, or pCMV; and most preferably, said expression vector is pcDNA3.

In preferred embodiments, said DNA vaccine may further comprise a pharmaceutically acceptable carrier. More preferably, said pharmaceutically acceptable carrier is ddH₂O or PBS (phosphate buffered saline).

In preferred embodiments, said DNA vaccine may be used as a pharmaceutical composition to treat HPV-related diseases; more preferably, to treat a genital cancer (e.g., cervical cancer, vaginal cancer, vulvar cancer, penile cancer) or a precancerous lesion (e.g., cervical, vulvar, or vaginal precancerous lesion), a head and neck cancer (e.g., oropharyngeal squamous cell carcinoma), a HPV-associated lung cancer, or a gastrointestinal cancer (e.g., esophageal cancer, or colorectal cancer); most preferably, used to treat cervical cancer.

The present invention further provides a method for preventing or treating a HPV-related disease, comprising administering an effective amount of the above-mentioned DNA vaccine to a subject having or at risk for developing said HPV-related disease.

In preferred embodiments, said HPV-related disease is a genital cancer or precancerous lesion, a head and neck cancer, or a gastrointestinal cancer; more preferably, said genital cancer comprises cervical cancer, lung, esophageal, nasopharyngeal, vaginal cancer, vulvar cancer, and penile cancer; said genital precancerous lesion comprises cervical, vulvar, and vaginal precancerous lesions; said head and neck cancer comprises oropharyngeal squamous cell carcinoma, and said gastrointestinal cancer comprises esophageal cancer, colorectal and anal cancer and precancerous lesions of the same; most preferably, said HPV-related disease is cervical cancer.

In summary, the present invention provides a DNA vaccine, which comprises a DNA construct comprising an expression vector which is expressible in a eukaryotic cell, an IL-6-encoding sequence and an E7(detox)-encoding sequence; which has an excellent potency and can be used for the treatment of HPV-related diseases.

### BRIEF DESCRIPTION OF THE DRAWINGS

None

### DETAILED DESCRIPTION OF THE PREFERRED EMBODIMENTS

HPV early genes are potentially useful as target antigens for therapeutic HPV vaccines because these early genes are expressed throughout the life cycle of HPV. The HPV early genes E6 and E7 (also known as oncogenes because of their ability to transform the host cells) are especially desirable for use in DNA vaccines to treat HPV-related diseases, including genital, head and neck, lung, and gastrointestinal cancers and related precancerous lesions. However, many DNA vaccines lack sufficient immunogenicity to render them useful because the DNA comprising the vaccines cannot amplify or spread in vivo, and antigen processing and presentation plays an important role in the production of useful therapeutic DNA vaccines.

Interleukin-6 (IL-6) is a cytokine expressed and secreted by normal cells, which plays an important role in the expansion and activation of T cells and in the differentiation of B cells. It also protects cells from apoptosis via the myeloid cell leukemia-1 (Mcl-1) pathway. These properties of IL-6 make it a desirable candidate for use in prolonging the lifespan of APCs, thereby improving the immune responses elicited by DNA vaccines in which it is a component.

The following examples are used to further illustrate the importance of the present invention, and are not intended to limit the claims of the invention. Of particular importance is that “E7” as stated in this invention refers to the E7 gene of any subtype of Human Pappilomavirus. The following mentioned “E7(detox)” is referred to the E7 gene with mutants in critical Rb binding residues which failed to bind Rb, had no transforming activity, and yet still maintained the potent immunogenicity. That is, the encoded amino acid of the E7(detox) presents the original HPV-E7 encoded amino acid with mutations or deletions at positions 24, 25 or 26. As one of the examples, the DNA sequence thereof is shown by SEQ ID NO:6. Specifically, the 70^{th} base “t” and the 77^{th} base “a” of E7 DNA sequence are respectively changed into “g”, both original HPV-E7 encoded amino acids at positions 24 and 26 are become into “Gly” in place of “Cys” and “Glu”, and finally E7(detox) is obtained. These substitutions have been shown to inhibit E7 protein binding to pRb at position 11, abating E7's ability to transform cells. Thus, it is more adequately used in clinical trials. In the following examples, the E7(detox) is shown by SEQ ID NO:6, but not limited thereto.

### EXAMPLES

### DNA Constructs and Preparation

IL-6 was amplified by polymerase chain reaction (PCR) using human placenta complementary DNA as the template and a set of primers, namely, 5′-CCGCTCGAGAGGAGCCCAGCTATGAACTC-3′ (SEQ ID NO: 1) and 5′-CCGGAATTCGACCAGAAGAAGGAATGCCC-3′ (SEQ ID NO: 2). The amplified IL-6 nucleotide sequence (SEQ ID NO: 3) was then cloned into the XhoI/EcoRI sites of pcDNA3 vector (Invitrogen, Carlsbad, Calif.) to generate pcDNA3-IL-6.

E7(detox) was amplified by PCR using the DNA of the CaSki cell line (a cell line containing an integrated HPV 16 genome, obtained from ATCC) as the template and with a set of primers, 5′-CCGGAAGCTTATGCATGGAGATACACCTAC-3′ (SEQ ID NO: 4) and 5′-CCCAAGCTTTTGAGAACAGATGG-3′ (SEQ ID NO: 5). The amplified E7(detox) nucleotide sequence (SEQ ID NO: 6) was then cloned into the HindIII sites of pcDNA3 and pcDNA3-IL-6 to generate pcDNA3-E7 and pcDNA3-IL-6/E7(detox) respectively. Said pcDNA3-IL-6/E7(detox) contains the IL-6 and E7(detox) nucleotide sequences joined in a continuous open reading frame such that a fusion protein comprised of IL-6 and E7(detox) are expressed by the construct. In addition, the E7 nucleotide sequence was also cloned into the HindIII sites of pcDNA3-Mcl-1 (a gift from Dr. H L Yen-Yang of Academia Sinica, Taiwan) to generate pcDNA3-Mcl-1/E7.

Next, pcDNA3-IL-6/E7(detox) or pcDNA3-Mcl-1/E7 was digested by HindIII, and then the GFP fragment obtained from the plasmid pcDNA3-E7/GFP (a gift from Dr. T C Wu of Johns Hopkins Medical Institutes) by EcoRI/NotI digestion filled and ligated to generate pcDNA3-IL-6/E7(detox)/GFP and pcDNA3-Mcl-1/E7/GFP. All DNA constructs were confirmed by DNA sequencing.

### Mice

Six- to eight-week-old female C57BL/6J mice were used in groups of five in all of the subsequently described studies. All animal procedures were performed according to approved protocols and in accordance with recommendations for the proper use and care of laboratory animals at the animal care facility of the College of Medicine, National Taiwan University.

### DNA Vaccination

Preparation of DNA-Coated Gold Particles and Particle-Mediated DNA Vaccination Using a helium-driven gene gun were performed by a low pressure-accelerated gene gun (BioWare Technologies Co. Ltd., Taipei, Taiwan). The gold particles (Bio-Rad 1652263) were weighted and suspended in 70% ethanol. This suspension was vortexed vigorously and then centrifuged to collect the particles. After washing with distilled water three times, 0.025 μg of the collected gold particles were put in an Eppendorf tube and mixed with 100 μl, of 0.05 M spermidine by vortexing, and the mixture was sonicated for 10 to 20 seconds. Subsequently, 25 μg of DNA dissolved in 25 μL of ddH₂O was added and the mixture was vortexed. 100 μL of 1 M CaCl₂ was added, and the final mixture was vortexed and incubated on ice for 10 minutes to coat DNA on the gold particles via spermidine. Finally, the coated particles were washed with 100% ethanol three times and resuspended in 200 to 250 μL of 100% ethanol. The DNA-coated gold particle suspension was used as a bullet for the gene gun. Naked DNA dissolved in ddH₂O or PBS also can be used as a bullet. DNA-coated gold particles were delivered to the shaved abdominal region of mice using the low pressure-accelerated gene gun with a 50 psi discharge pressure of helium.

### Intracellular Cytokine Staining and Flow Cytometric Analysis

Each group of mice was immunized with one of the following DNA vaccines: pcDNA3 (no insert), pcDNA3-E7, pcDNA3-IL-6, pcDNA3-E7(detox)+pcDNA3-IL-6, pcDNA3-Mcl-1/E7 and pcDNA3-IL-6/E7(detox) (2 μg DNA construct/2 μg gold particle/mouse); all mice received a booster immunization one week later. A DNA-naïve group, in which the mice were not vaccinated, was used as a negative control. One week after boosting, the mice were sacrificed and their splenocytes were harvested and incubated with either 1 μg/ml of short E7(detox) peptide RAHYNIVTF (aa 49-57, SEQ ID NO: 7) or 10 μg/ml of long E7(detox) peptide DSSEEEDEIDGPAGQAEPDRA HYNIVTFCCKCDSTLRL (aa 30-67, SEQ ID NO: 8). Generally speaking, the short E7(detox) peptide could be presented directly; however, the long E7(detox) peptide required up-take by the APCs first for subsequent processing and presentation. The cells were mixed with the short or long E7(detox) peptide for 16 to 20 hours. Next, 1 μL/mL Golgistop (PharMigen, San Diego, Calif.) was added to prevent the secretion of cytokines, such as IFN-γ or IL-4. Six hours later, cells were harvested, transferred into a tube, and then centrifuged at 1,200-1,600 rpm for 5 min at 4° C. Next, the cells were washed with 500 μL FACScan buffer (0.5% BSA in PBS), and re-centrifuged for 5 min at 4° C. The cells were re-suspended in 1 μL (0.5 ug) PE-conjugated anti-CD4 or anti-CD8 Ab (PharMingen) diluted in 50 μL FACScan buffer, and the cells, were incubated for 30 minutes in the dark. The cells were then washed twice with FACScan buffer and centrifuged. These cells were re-suspended in 500 μL fixation buffer on ice for 20 minutes in the dark; subsequently, the cells were again centrifuged and washed with 500 μL Perm Wash buffer (BioLegend Biotech). 1 μL (0.5 ug) FITC-conjugated anti-IFN-γ Ab (PharMingen) or anti-IL-4 Ab (Biolegend, San Diego, Calif.) was diluted in 50 μL Perm Wash buffer, added to the cells, and incubated on ice for 30 minutes in the dark. The cells were centrifuged and washed twice with 500 μL Perm Wash buffer. Then the cells were resuspended in 300 to 500 μL FACScan buffer, and analyzed by flow cytometry. All double-stained cells are analyzed by FACScan or FACSCalibur with CELLQuest software (Becton Dickinson Immunocytometry System, Mountain View, Calif., USA) using a standard procedure. The results are shown in Table 1.

Table 1 shows the numbers of E7(detox)-specific IFN-γ-secreting CD4⁺ T lymphocytes, E7(detox)-specific IFN-γ-secreting CD4⁺ T lymphocytes and E7(detox)-specific IL-4-secreting CD4⁺ T lymphocytes per 3.5×10⁵ splenocytes, which are analyzed by the flow cytometric analysis. These data show that the group of mice vaccinated with IL-6/E7(detox) generated more E7(detox)-specific IFN-γ-secreting CD4⁺ T lymphocytes, E7(detox)-specific IL-4-secreting CD4⁺ T lymphocytes and E7(detox)-specific IFN-γ-secreting CD8⁺ T lymphocytes than other groups. In other words, IL-6/E7(detox) could activate Th1 pathway and Th2 pathway.

Regardless which E7 peptide was used in these experiments, the numbers of CD4⁺ IFN-γ-secreting and CD8⁺ IFN-γ-secreting E7-specific T lymphocytes was significantly higher in the IL6/E7(detox) vaccinated mice as compared to those vaccinated with E7 alone.

TABLE 1

| | Flow Cytometry | | | | | |
|---|---|---|---|---|---|---|
| | IFN-γ CD4⁺ T | | IL-4 CD4⁺ T | | IFN-γ CD8⁺ T | |
| | lymphocytes | | lymphocytes | | lymphocytes | |
| | E7: 30-67 aa | | E7: 30-67 aa | | E7: 49-57 aa | |
| | mean | SD | mean | SD | mean | SD |
| Naïve | 8 | 2.1 | 16 | 2.8 | 4 | 2.1 |
| No Insert | 12.5 | 2.1 | 22.5 | 2.1 | 6 | 2.1 |
| E7 | 11 | 2.1 | 22 | 3.5 | 6.5 | 1.4 |
| IL-6 | 5.5 | 1.4 | 21.5 | 2.1 | 4 | 1.4 |
| IL-6 + E7 | 6.5 | 1.4 | 26.5 | 2.8 | 9 | 2.8 |
| (detox) | | | | | | |
| IL-6/E7 (detox) | 77 | 12 | 292 | 16.5 | 432.5 | 15.4 |
| p-value | p < 0.05 | | p < 0.01 | | p < 0.01 | |

### Enzyme-Linked Immunosorbent Assay (ELISA) for Anti-E7 Antibodies

Sera from all mice were extracted 14 days after the last immunization and were used to detect HPV-16 E7-specific antibodies by the direct enzyme-linked immunosorbent assay described below.

A 96-microwell plate was coated with 100 μL of bacteria-derived HPV-16 E7(detox) protein (0.5 μg/ml) and incubated at 4° C. overnight. The wells were then blocked with PBS containing 20% fetal bovine serum at 37° C. for 2 hours. 100 μL of the sera at various dilutions of 1:100, 1:500, or 1:1,000 in PBS was then added and the plate was incubated at 37° C. for 2 hours. The wells were then washed with PBS containing 0.05% Tween 20, and then incubated with a 1:2000 dilution of peroxidase-conjugated rabbit anti-mouse IgG antibody (Zymed, San Francisco, Calif.) at room temperature for 1 hour. The plate was washed, developed with 1-Step Turbo TMB-ELISA (Pierce, Rockford, Ill.), and the reaction was stopped with 1M H₂SO₄. The ELISA plate was read with a standard ELISA reader at 450 nm.

Table 2 shows E7-specific antibodies in mice vaccinated with the various DNA vaccines. Mice vaccinated with the IL-6/E7(detox) DNA vaccine had a titer higher than those in the other groups.

TABLE 2

ELISA - Antibody to E7
1:100 dilution

| | mean | SD |
|---|---|---|
| Naïve | 0.434 | 0.027 |
| No Insert | 0.418 | 0.021 |
| E7 | 0.454 | 0.037 |
| IL-6 | 0.403 | 0.025 |
| IL-6 + E7(detox) | 0.415 | 0.016 |
| IL-6/E7(detox) | 1.063 | 0.05 |
| p-value | p < 0.01 | |

### In Vivo Tumor Protection Experiments

To determine whether the observed enhancement of E7(detox)-specific T cell immunity can be translated into a significant E7-specific protective anti-tumor effect, we performed the following in vivo tumor protection experiments. Each group of mice was immunized with pcDNA3 (no insert), pcDNA3-E7, pcDNA3-IL-6, pcDNA3-E7(detox)+pcDNA3-IL-6, pcDNA3-Mcl-1/E7 or pcDNA3-IL-6/E7(detox) (2 μg/mouse), and all mice received a booster one week later. Mice were challenged with 5×10⁴ TC-1 cells/mouse by subcutaneous injection one week after the boosting. These mice were monitored until 60 days after TC-1 challenge. The naïve group, in which the mice were not vaccinated, was used as a negative control.

TC-1 cells were grown in RPMI-1640 supplemented with 10% (vol/vol) fetal bovine serum, 50 units/mL penicillin/streptomycin, 2 mM L-glutamine, 1 mM sodium pyruvate, 2 mM non-essential amino acids (Gibco company) and 0.4 mg/mL G418 at 37° C. with 5% CO₂. On the day of tumor challenge, TC-1 cells were harvested by trypsinization, washed twice with 1×PBS and finally re-suspended in 1× Hanks buffered salt solution for TC-1 challenge.

After challenging with TC-1 tumor cells, 100% of the mice that received the IL-6/E7(detox) DNA vaccine remained tumor-free at day 60, which indicates 100% protection. Only 40% of the mice that received the Mcl-1/E7 DNA vaccine remained tumor-free at day 60, and all mice in the other groups, including the E7 group, developed tumors within 14 days after TC-1 challenge. The results are shown in Table 3.

TABLE 3

| | Tumor free |
|---|---|
| Naïve | 0% |
| No Insert | 0% |
| E7 | 0% |
| IL-6 | 0% |
| IL-6/E7(detox) | 100% |

### In Vivo Antibody Depletion Experiment

To determine whether the subsets of lymphocytes (CD4⁺ T lymphocytes, CD8⁺ T lymphocytes and NK lymphocytes) are important for the anti-tumor effects, we performed an in vivo antibody depletion experiment. The mice were vaccinated with IL-6/E7(detox) DNA vaccine, boosted one week later, and then subjected to lymphocyte subset depletion. Monoclonal Ab GK1.5 was used for CD4 depletion (Berkeley Antibody Company), monoclonal Ab 2.43 for CD8 depletion (Berkeley Antibody Company), and monoclonal Ab PK136 for Natural Killer 1.1⁺ depletion (Berkeley Antibody Company).

The monoclonal antibodies were injected intraperitoneally. One week after depletion, all groups of mice were challenged with 5×10⁴ TC-1 cells/mouse. Flow cytometric analysis revealed that 99% of these lymphocyte subsets were depleted while the other subsets were maintained at normal levels (data not shown). All mice were terminated on day 40 after the tumor challenge.

All naïve mice and all mice depleted of CD8+ T cells grew tumors within 14 days after TC-1 challenge. In addition, 20% of mice depleted of CD4+ T cells and 40% of mice depleted of natural killer 1.1+ cells developed tumors within 60 days after TC-1 challenge. The results are shown in Table 4, which suggests that CD4⁺ T cells are important for the anti-tumor immunity generated by the IL-6/E7(detox) DNA vaccine.

TABLE 4

| | | CD4⁺ | CD8⁺ | |
|---|---|---|---|---|
| | No depletion | depletion | depletion | NK depletion |
| | Tumor free | | | |
| IL-6/E7 (detox) | 100% | 80% | 0% | 60% |

### In Vivo Tumor Treatment Experiment

The therapeutic potential of the chimeric IL-6/E7(detox) DNA vaccine was assessed by a lung hematogenous spread model because of its similarity to cancer metastasis (see Cheng et al., 2005a). In this experiment, mice were injected with 5×10⁴ TC-1 cells via their tail veins. Two days later, each group of mice was immunized with pcDNA3 (no insert), pcDNA3-E7, pcDNA3-IL-6, pcDNA3-E7(detox)+pcDNA3-IL-6, pcDNA3-Mcl-1/E7 or pcDNA3-IL-6/E7(detox) (16 μg/mouse), followed by a booster immunization every 7 days. The mice were sacrificed and their lungs were removed 28 days after TC-1 challenge. The pulmonary tumor nodules in each mouse were evaluated and counted by experimenters who were blind to the sample identity. The naïve group, in which the mice were not vaccinated, was used as a negative control.

Referred to Table 5, which is the mean of lung weight and the number of pulmonary tumor nodules of each vaccinated mice group. Mice vaccinated with IL-6/E7(detox) had much lower lung weights in comparison to mice in the other groups, and they also had significantly fewer pulmonary tumor nodules than all the other groups.

TABLE 5

| | |
|---|---|
| Lung Weight (mg) | Number of Tumor |

| | mean | SD | mean | SD |
|---|---|---|---|---|
| E7 | 630 | 19.5 | 87.6 | 7.4 |
| IL-6/E7 (detox) | 153 | 18.2 | 1.5 | 1.2 |
| p-value | p < 0.001 | | p < 0.001 | |

### Cytotoxic T-Lymphocyte (CTL) Assays Using CaSki Cells as Target Cells

Peripheral blood mononuclear cells were obtained from HLA-A2-positive human volunteers and were induced to differentiate into dendritic cells (DCs) by exposure to granulocyte macrophage colony-stimulating factor (GM-CSF) (800 U/ml) and IL-4 (500 U/ml) for 6 days. The differentiated DCs were grouped and each group was pulsed with 50 mmol/l of cell lysates obtained from 293 D^{b}K^{b} cells transfected with pcDNA3-E7 or pcDNA3-IL-6/E7(detox) DNA constructs overnight, and each group of pulsed DCs was then incubated with fresh peripheral blood mononuclear cells from the same human volunteers to generate four sets of E7(detox)-specific CD8⁺ T cells. CTL assays were then performed wherein each E7(detox)-specific CD8⁺ T cell sets were used as effector cells and CaSki cells were used as the target cells. The cytotoxic T cell activity (CTL) level was measured using a standard lactate dehydrogenase (LDH) release assay (see Cheng et al., 2005b). Effector cells and target cells (1×10⁴ target cells per well) were mixed together at a variety of ratios (1:1, 5:1, 15:1 and 45:1) in a final volume of 200 μL. The cell mixtures were incubated for 5 hours at 37° C., and then 50 μL of the culture medium was collected to assess the quantity of LDH in the culture medium and count the percentage of cell lysis according to the protocol of the CytoTox assay kits (Promega, Madison, Wis.). All experiments were performed in triplicate.

As shown in Table 6, the percentage of specific lysis of CaSki cells obtained from IL-6/E7(detox) protein-pulsed cytotoxic T cells was significantly higher than that obtained from E7 protein-pulsed E7-specific cytotoxic T cells.

TABLE 6

| % of Specific Lysis | |
|---|---|
| E:T = 45:1 | 50 μg/mL |

| | mean | SD | mean | SD |
|---|---|---|---|---|
| E7 | 9.4 | 1.3 | 8.8 | 0.3 |
| IL-6/E7(detox) | 57.3 | 6.5 | 45.5 | 2 |
| p-value | p < 0.01 | | p < 0.01 | |

### Statistical Analysis

All data expressed as mean±SEM are representative of at least two different experiments. Data for intracellular cytokine staining with flow cytometric analysis and tumor treatment experiments were evaluated by analysis of variance (ANOVA).

What is claimed is:

1. A DNA construct, consisting of:
an expression vector, which is expressible in a eukaryotic cell;
an Interleukin-6 (IL-6)-encoding sequence; and
a Human papillomavirus E7(detox) (HPV E7(detox))-encoding sequence;
wherein the IL-6-encoding sequence consists of SEQ ID NO: 3, and the encoded amino acid of the HPV E7(detox)-encoding sequence presents an original HPV-E7 encoded amino acid with mutations or deletions at positions 24, 25 or 26 which disrupt a retinoblastoma (Rb) binding site.

2. The DNA construct according to claim 1, wherein the encoded amino acid of the HPV E7(detox)-encoding sequence presents the original HPV-E7 encoded amino acid with mutations at positions 24 and 26.

3. The DNA construct according to claim 2, wherein the HPV E7(detox)-encoding sequence consists of SEQ ID NO: 6.

4. The DNA construct according to claim 1, wherein said expression vector is expressible in human cells.

5. The DNA construct according to claim 4, wherein said expression vector is selected from pcDNA3, pSG5, or pCMV.

6. A DNA vaccine, comprising the DNA construct according to claim 1.

7. The DNA vaccine according to claim 6, further comprising a particle, which is coated with said DNA construct.

8. The DNA vaccine according to claim 7, wherein said particle is a gold particle.

9. The DNA vaccine according to claim 8, wherein said gold particle has a diameter in a range of 1.6 μm.

10. The DNA vaccine according to claim 6, wherein said expression vector is expressible in human cells.

11. The DNA vaccine according to claim 10, wherein said expression vector is selected from pcDNA3, pSG5, or pCMV.

12. The DNA vaccine according to claim 6, further comprising a pharmaceutically acceptable carrier.

13. A method for preventing or treating a HPV-related disease, comprising:
administering an effective amount of the DNA vaccine according to claim 6 to a subject having or at risk for developing said HPV-related disease.

14. The method according to claim 13, wherein said HPV-related disease is cervical cancer, vaginal cancer, vulvar cancer, lung cancer, esophageal cancer, nasopharyngeal cancer (NPC), or penile cancer.

15. The method according to claim 14, wherein said HPV-related disease is a cervical, lung, esophageal, nasopharyngeal, vulvar, or vaginal precancerous lesion.


SEQUENCE LISTING (8 sequences)

SEQ ID NO: 1 - DNA, 29 bp, Artificial
  primer
ccgctcgagaggagcccagctatgaactc

SEQ ID NO: 2 - DNA, 29 bp, Artificial
  primer
ccggaattcgaccagaagaaggaatgccc

SEQ ID NO: 3 - DNA, 700 bp, Human
aggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtc

SEQ ID NO: 4 - DNA, 30 bp, Artificial
  primer
ccggaagcttatgcatggagatacacctac

SEQ ID NO: 5 - DNA, 23 bp, Artificial
  primer
cccaagcttttgagaacagatgg

SEQ ID NO: 6 - DNA, 288 bp, human papillomavirus 16
atgcatggagatacacctacattgcatgaatatatgttagatttgcaaccagagacaactgatctctacggttatgggcaattaaatgacagctcagaggaggaagatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaa

SEQ ID NO: 7 - protein, 9 aa, human papillomavirus 16
RAHYNIVTF

SEQ ID NO: 8 - protein, 38 aa, human papillomavirus 16
DSSEEEDEIDGPAGQAEPDRAHYNIVTFCCKCDSTLRL